(19) United States
(12) Patent Application Publication
(10) Pub. No.: US 2025/0245787 A1
(43) Pub. Date: Jul. 31, 2025

(54) X-RAY IMAGING METHOD, DATA PROCESSING APPARATUS, X-RAY IMAGING SYSTEM, AND COMPUTER PROGRAM PRODUCT

(71) Applicant: Siemens Healthineers AG, Forchheim (DE)

(72) Inventor: Michael Manhart, Fürth (DE)

(21) Appl. No.: 19/027,148

(22) Filed: Jan. 17, 2025

(30) Foreign Application Priority Data
Jan. 31, 2024 (EP) 24154923

(51) Int. Cl. G06T 5/50; A61B 6/00; A61B 6/46; A61B 6/50; G06T 5/60; G06T 7/33; G16H 30/40

(52) U.S. Cl. CPC G06T 5/50; A61B 6/463; A61B 6/481; A61B 6/504; G06T 5/60; G06T 7/337; G16H 30/40; G06T 2207/10116; G06T 2207/20081; G06T 2207/20084; G06T 2207/20224; G06T 2207/30101

(57) ABSTRACT

In an X-ray imaging method, at least two X-ray images are obtained of a region to be depicted in an object, wherein the at least two X-ray images correspond to different acquisition timeframes and represent a vascular structure of the object in different contrast-agent filling states. A shared vessel mask, which represents the vascular structure, is generated based on the at least two X-ray images. At least two live images of the region to be depicted are obtained, which correspond to different second acquisition timeframes. For each image of the at least two live images, depending on the shared vessel mask, a corresponding overlay image for displaying on a display device is generated.

The present patent document claims the benefit of European Patent Application No. 24154923, filed Jan. 31, 2024, which is hereby incorporated by reference in its entirety.

### TECHNICAL FIELD

The present disclosure relates to an X-ray imaging method, wherein at least two X-ray images are obtained of a region to be depicted in an object, and wherein the at least two X-ray images correspond to different first acquisition timeframes and represent a vascular structure of the object in different contrast-agent filling states. The disclosure also relates to a data processing apparatus for performing the X-ray imaging method, to a corresponding X-ray imaging system, and to a corresponding computer program product.

### BACKGROUND

In vascular interventions, under X-ray monitoring, vessel masks may be overlaid on live images during the vascular intervention in order to enhance in the resultant overlay image a corresponding vascular structure for the treatment personnel, for instance in order to assist the personnel in guiding a medical instrument in the vascular structure. In particular, methods for digital subtraction angiography, DSA, and/or roadmap methods may be carried out for this purpose.

Movements of the object to be depicted, (e.g., the patient), which are caused, for instance, by the patient breathing and/or organ movements and reduce the accuracy of the overlay, are a problem in these methods.

One possible way of counteracting this is to instruct the patient accordingly to hold his or her breath during the acquisition of X-ray images. This is not only unpleasant for the patient, however, but also relatively unreliable. Moreover, this cannot prevent movements caused by the heartbeat or other organ movements.

Document DE 10 2021 208 272 A1 proposes a method for generating a subtraction image for DSA in order to reduce noise and motion artifacts. In this method, a plurality of mask images of an object is obtained before a contrast agent is administered into the object, and a depiction of the object after administration of the contrast agent. A first summation image is obtained from the plurality of mask images by summing the plurality of mask images, each multiplied by an individual weight. The individual weights for each of the plurality of mask images are ascertained automatically by an optimization method, and the subtraction image is determined by subtracting the summation image from the depiction.

This, however, only addresses the movement that occurs while the mask images are being generated but not movements during generation of a plurality of depictions or live images in succession.

The publication by O. Ronneberger et al: “U-Net: Convolutional Networks for Biomedical Image Segmentation” (arXiv: 1505.04597) describes the U-Net architecture, a widely established CNN architecture for image segmentation but which may also be used for other computer vision tasks such as object recognition et cetera.

The publication J. Chen et al.: “TransUNet: Transformers Make Strong Encoders for Medical Image Segmentation” (arXiv: 2102.04306) describes TransUNet, which combines transformer networks with U-Net for the purpose of medical-image segmentation.

The publication H. Wang et al.: “Mixed Transformer U-Net For Medical Image Segmentation” describes a transformer module named Mixed Transformer Module, MTM, for medical-image segmentation.

### SUMMARY AND DESCRIPTION

An object of the present disclosure is to reduce the effects of movements of an object to be depicted when a plurality of live images, acquired in different acquisition timeframes, are meant to be overlaid with vessel masks.

The scope of the present disclosure is defined solely by the appended claims and is not affected to any degree by the statements within this summary. The present embodiments may obviate one or more of the drawbacks or limitations in the related art.

The disclosure is based on generating a shared vessel mask on the basis of at least two X-ray images and generating a corresponding overlay image based on the shared vessel mask for each of at least two live images.

According to one aspect, an X-ray imaging method is specified. In this method, at least two X-ray images are obtained of a region to be depicted in an object, wherein the at least two X-ray images correspond to different first acquisition timeframes and each represent a vascular structure of the object in different contrast-agent filling states. A shared vessel mask, which represents the vascular structure, is generated on the basis of the at least two X-ray images. At least two live images of the region to be depicted are obtained, which correspond to different second acquisition timeframes. For each of the at least two live images, depending on the shared vessel mask, a corresponding overlay image for displaying on a display device is generated.

The X-ray imaging method may be purely computer-implemented. Unless stated otherwise, all the acts of the computer-implemented method may be performed by a data processing apparatus that includes at least one computing unit. In particular, the at least one computing unit is configured or adapted to execute the acts of the computer-implemented method. For this purpose, the at least one computing unit may store, for example, a computer program containing commands that, when executed by the at least one computing unit, cause the at least one computing unit to execute the computer-implemented method.

In the case that the at least one computing unit includes two or more computing units, certain acts implemented by the at least one computing unit may also be understood in the sense that different computing units implement different acts or different parts of an act. In particular, it is not necessary for each computing unit to implement the acts in full. In other words, the implementation of the acts may be distributed over the two or more computing units.

From each embodiment of the computer-implemented method, a corresponding embodiment of an X-ray imaging method, which is not purely computer-implemented, is obtained by incorporating corresponding acts for generating the at least two X-ray images and/or the at least two live images.

Unless stated otherwise, an image may be understood to mean here and below an X-ray projection image or a preprocessed X-ray projection image or a combination of X-ray projection images or preprocessed X-ray projection images.

The first acquisition timeframes are in particular consecutive acquisition timeframes, which may have the same duration, for example. Each of the at least two X-ray images is generated during one of the first acquisition timeframes, in particular by an X-ray imaging system.

The at least two X-ray images may be contrast-agent images, e.g., images that were generated after or during administration of a contrast agent. The at least two X-ray images may also be subtraction images, which were generated in particular using DSA. For example, corresponding contrast-agent images are set against one or more mask images generated without administration of contrast agent in order to enhance the vascular structure more clearly.

As a result of the time dynamics of the distribution of the contrast agent in the vascular structure, each of the at least two X-ray images shows another contrast-agent filling state of the vascular structure. In particular, in different X-ray images of the at least two X-ray images, different regions of the vascular structure may be filled with contrast agent, and/or the amount of contrast agent in a certain region of the vascular structure may vary for different X-ray images of the at least two X-ray images, and/or a total amount of contrast agent in the vascular structure may vary for different X-ray images of the at least two X-ray images.

The second acquisition timeframes are in particular consecutive acquisition timeframes, which may have the same duration, for example. Each live image of the at least two live images is generated during one of the second acquisition timeframes, in particular by the X-ray imaging system. The at least second acquisition timeframes may lie after the first acquisition timeframes. For example, the at least two live images are generated without administration of contrast agent. The live images may be generated, for example, during a vascular intervention on the vascular structure.

The overlay images may be displayed on the display device, in particular successively according to the sequence of the at least two live images.

For example, the shared vessel mask may correspond to a monochromatic image, for instance a grayscale image, in which the grayscale value of a pixel represents the corresponding amount of contrast agent in the corresponding region of the vascular structure. It may also be a binary image, in which pixels having a first binary value, (e.g., 1), belong to the vascular structure, and pixels having a second binary value, (e.g., 0), do not belong to the vascular structure or may not be associated with the vascular structure based on the at least two X-ray images. In particular, the vessel mask does not show any parts, or shows as few parts as possible, of the region to be depicted that does not correspond to the vascular structure.

Depending on the embodiment of the X-ray imaging method, the shared vessel mask may correspond to precisely one of the different contrast-agent filling states. The shared vessel mask may also correspond to a virtual contrast-agent filling state, however, which is thus not represented by any of the at least two X-ray images.

The shared vessel mask may be generated in different ways in different embodiments. One possible way is to select one of the at least two X-ray images, (e.g., one having a maximum amount of contrast agent in the vascular structure), and to extract therefrom, (e.g., by known vessel segmentation methods or by other methods for vessel extraction, for instance based on the use of a trained machine-learning model), the vascular structure or parts thereof in order to generate the shared vessel mask. It is also possible to generate on the basis of the at least two X-ray images, or representations of the vascular structure that are extracted therefrom, a representation of the virtual contrast-agent filling state, (e.g., using a trained machine-learning model). It is also possible to extract on the basis of the at least two X-ray images a representation of the vascular structure in each case, and to combine these, (e.g., using a trained machine-learning model), in order to generate the shared vessel mask.

Each of the overlay images is generated on the basis of the same shared vessel mask and on the basis of the corresponding live image. This does not necessarily imply, however, that each of the live images is overlaid with the shared vessel mask in the same way. In particular, it is also possible that a variant of the shared vessel mask is generated for each live image, (e.g., by spatial displacement of the shared vessel mask), and this variant of the shared vessel mask is overlaid on the associated live image.

By generating an individual overlay image for each live image, it is possible to compensate, at least in part, for effects of movements of the object during the generation of the live images. Because all the overlay images are generated on the basis of the same shared vessel mask, not only is the vascular structure represented consistently in all the overlay images, but also the computational effort may be reduced overall.

The overlay image may be generated by overlaying on the live image the shared vessel mask or the associated variant of the shared vessel mask. It is also possible, however, to generate a further subtraction image on the basis of the live image, and the overlay image may be generated by overlaying on the further subtraction image the shared vessel mask or the associated variant of the shared vessel mask. The further subtraction image may be generated, for example, by subtracting a further mask image from the live image.

For example, the live image may represent the region to be depicted containing an instrument, for instance a catheter, a vascular balloon, a stent, a guide wire or the like, and the further mask image may represent the region to be depicted without the instrument. Thus, the instrument is enhanced in the further subtraction image compared with the live image. The term “roadmap method” may be used in this case, for example, in particular if it involves generating the subtraction images.

According to at least one embodiment, at least one mask image of the region to be depicted and at least two contrast-agent images of the region to be depicted are obtained, wherein the at least two contrast-agent images represent the vascular structure in the different contrast-agent filling states. The at least two X-ray images are given by the at least two contrast-agent images.

This reduces the effort involved in generating the at least two X-ray images, with regard to both the effort for generating the raw data by an X-ray imaging system and the computational effort for generating the at least two X-ray images on the basis of the raw data.

According to at least one embodiment, at least one mask image of the region to be depicted and at least two contrast-agent images of the region to be depicted are obtained, wherein the at least two contrast-agent images represent the vascular structure in the different contrast-agent filling states. For each contrast-agent image of the at least two contrast-agent images, a subtraction image is generated on the basis of the at least one mask image. The at least two X-ray images are given by the subtraction images.

The at least two contrast-agent images here correspond to the different first acquisition timeframes and represent the vascular structure in the different contrast-agent filling states.

This achieves that the vascular structure in the at least two X-ray images is enhanced more clearly, which increases the accuracy of the shared vessel mask and hence of the overlay images.

The at least one mask image may be precisely one mask image. The subtraction images may then be generated, for example, by subtracting the mask image from the respective contrast-agent image. This reduces the effort involved in generating the at least one mask image, both the effort for generating the raw data by an X-ray imaging system and the computational effort for generating the at least one mask image on the basis of the raw data.

The at least one mask image may also include two or more mask images, for example, a mask image for each of the contrast-agent images. The particular subtraction image is then generated by subtracting the associated mask image from the corresponding contrast-agent image. This allows the vascular structure to be represented more accurately in the subtraction images.

According to at least one embodiment, the at least one mask image contains at least two mask images, wherein the at least two mask images correspond to different further acquisition timeframes. For each of the at least two contrast-agent images, a corresponding summation image (9a, 9b) is generated by weighted summation of the at least two mask images. For each of the at least two contrast-agent images, the associated subtraction image is generated by subtracting the associated summation image (9a, 9b) from the particular contrast-agent image.

Corresponding weighting factors for generating the summation image (9a, 9b) may be ascertained automatically by an optimization method, for example. In particular, a method as described in the document DE 10 2021 208 272 A1 cited in the introduction may be used, in particular by solving an optimization problem as defined in paragraph of the cited document.

This may increase the accuracy of the representation of the vascular structure in the subtraction images, in particular may at least partially compensate for movement of the object during generation of the mask images and/or the contrast-agent images. This consequently also increases the accuracy of the representation of the vascular structure in the shared vessel mask and in the overlay images.

In particular, the at least two mask images may be the same in number as the at least two contrast-agent images. The further acquisition timeframes may lie before the first acquisition timeframes, for example, in particular before corresponding administration of contrast agent.

According to at least one embodiment, the generating of the shared vessel mask includes applying at least one trained machine-learning model, MLM, to input data, which depends on the at least two X-ray images, for instance includes these or is generated on the basis of thereof. The output from the trained machine-learning model includes in particular the shared vessel mask, or the shared vessel mask is generated depending on the output from the at least one trained MLM.

A trained MLM may mimic cognitive functions that humans associate with another human mind. In particular, as a result of training based on training data, the MLM is capable of adapting to new circumstances and detecting and extrapolating patterns. Another term for a trained MLM is “trained function.”

The parameters of an MLM may be adapted or updated by training. In particular, the terms supervised training, semi-supervised training, unsupervised training, reinforcement learning, and/or active learning may be used here. It is also possible to use representation learning, also known as feature learning. In particular, the parameters of the MLMs are adapted iteratively by a plurality of training acts. In particular, a specific loss function, also known as a cost function, may be minimized in the training. The backpropagation algorithm, in particular, may be used in the training of an artificial neural network, ANN.

An MLM may include an ANN, a support vector machine, a decision tree, and/or a Bayes network, and/or the MLM may be based on k-means clustering, Q-learning, genetic algorithms, and/or association rules. In particular, an ANN may be, or may include, a deep neural network, a convolutional neural network, CNN, or a deep convolutional neural network. In addition, an ANN may be an adversarial network, a deep adversarial network, and/or a generative adversarial network.

According to at least one embodiment, the generating of the shared vessel mask includes applying a trained first machine-learning model to first input data, which depends on the at least two X-ray images.

For example, the first MLM may be an ANN, in particular a CNN, for instance a U-Net or an ANN based on a U-Net.

The output from the trained first MLM includes in particular the shared vessel mask, or the shared vessel mask is generated depending on the output from the trained first MLM.

In some embodiments, the input data may contain the at least two X-ray images, in particular if there is no provision to generate the subtraction images.

In embodiments that provide for generating the subtraction images, the first input data may depend on, in particular include, the subtraction images, for example. Alternatively, a combination of the particular subtraction image with the associated contrast-agent image may be generated for each of the subtraction images, for instance by weighted summation. In the resultant combination, the vascular structure is particularly enhanced in comparison with the associated contrast-agent image. For example, the first input data may depend on or include the combinations.

For example, the first MLM may be trained to predict the shared vessel mask on the basis of all the X-ray images of the at least two X-ray images. The shared vessel mask may represent, for example, the vascular structure extracted from one of the X-ray images, for instance from that in which the vascular structure is optimally filled, in particular filled to a maximum, with the contrast agent. Alternatively, the shared vessel mask may represent the vascular structure in the virtual contrast-agent filling state, which the first MLM predicts on the basis of all of the at least two X-ray images.

According to at least one embodiment, for each X-ray image of the at least two X-ray images a provisional vessel mask is generated by applying a trained first MLM to first input data, which depends on the particular X-ray image. The shared vessel mask is generated depending on the provisional vessel masks.

The provisional vessel masks may represent, for example, the vascular structure in the contrast-agent filling state of the associated X-ray image. Thus, the first MLM is trained in particular to extract the vascular structure from the associated X-ray image, in particular when there is no provision to generate the subtraction images. In embodiments that provide for generating the subtraction images, the first input data may depend on, in particular include, the subtraction images, for example. The first MLM is then trained in particular to extract the vascular structure from the associated subtraction image. Alternatively, for each of the subtraction images, a combination of the particular subtraction image with the associated contrast-agent image may be generated for instance by weighted summation. In the resultant combination, the vascular structure is particularly enhanced in comparison with the associated contrast-agent image. For example, the first input data may depend on or include the combinations. The first MLM is then trained in particular to extract the vascular structure from the particular combination.

Using the first MLM has the advantage, for example, that the first MLM may be employed universally and may extract the vascular structure with high accuracy.

In some embodiments, another method as an alternative to the first MLM may also be used, in particular known per se, for vessel extraction or vessel segmentation, in particular for generating the provisional vessel masks. In particular, this may save the effort involved in training an MLM.

According to at least one embodiment, the shared vessel mask is selected as one of the provisional vessel masks according to a defined rule.

The defined rule may specify, for example, that the best, according to defined criteria, of the provisional vessel masks is selected. For example, the best provisional vessel mask may be the one that corresponds to the contrast-agent filling state that has the largest total amount of contrast agent. The total amount of contrast agent may be ascertained, for example, by calculating an L2 norm of the particular provisional vessel mask or another suitable metric.

This achieves that for each overlay image the contrast-agent filling state that has the largest total amount of contrast agent is represented. The medical personnel may thus be provided with the maximum amount of available information.

According to at least one embodiment, the shared vessel mask is generated by applying a trained second MLM to second input data, which depends on, in particular includes, the provisional vessel masks.

The second MLM may be trained to predict the provisional vessel mask as the shared vessel mask in accordance with the defined rule. It is thereby possible to implement in particular more complex rules, for instance relating to the spatial distribution of the contrast agent in the vascular structure or sufficient filling of certain critical parts of the vascular structure by the contrast agent.

The second MLM may also be trained to predict the shared vessel mask from the provisional vessel masks such that it represents the virtual contrast-agent filling state. This may improve further the information provided to the medical personnel.

According to at least one embodiment, a registration instruction is determined for each provisional vessel mask of the provisional vessel masks depending on the shared vessel mask. For each of the at least two live images, one of the at least two X-ray images is identified by comparing the particular live image with the at least two X-ray images. For each of the at least two live images, for the purpose of generating the overlay image, the vessel mask is registered with the particular live image by the registration instruction that corresponds to the identified X-ray image and is overlaid on the particular live image.

The registration instruction may include a displacement vector, for example. If the pixels in the shared vessel mask are displaced by the displacement vector, this results in a displaced vessel mask that matches, at least in parts, the associated provisional vessel mask. For example, the registration instruction may include a corresponding displacement vector for each pixel of the shared vessel mask or for each pixel group of a multiplicity of predefined pixel groups of the shared vessel mask.

The registration instruction may be ascertained by an optimization method, for example, which minimizes a metric for quantifying a deviation between the shared vessel mask and the particular provisional vessel mask or maximizes a metric for quantifying a match between the shared vessel mask and the particular provisional vessel mask.

For example, the registration instruction may be ascertained by maximizing a normalized cross correlation, NCC, between the shared vessel mask and the particular provisional vessel mask. Alternatively, a registration technique based on gradient optimization may be used, or a further trained MLM may be used for the registration.

If the at least two X-ray images are denoted by Rᵢ, where i=1, . . . , N with N≥2 is the index for the individual X-ray images, then the provisional vessel masks may be denoted by VMᵢ and the associated registration instructions by Pᵢ. Thus, each i=1, . . . , N is associated with one VMᵢ, one Rᵢ and one Pᵢ. Analogously, the at least two live images may be denoted by Lₖ, where k=1, . . . , M with M≥2.

A given live image Lₖ is then compared in particular with all the X-ray images Rᵢ, in particular by calculating a suitable metric that states the similarity between Lₖ and Rᵢ, for instance the NCC. The Rᵢ that has the best match with the given Lₖ is identified, so for example the one that has the largest metric, for instance L2 norm. The associated i may be denoted by i*.

In order to generate the overlay image, the shared vessel mask is then displaced pixel by pixel according to the registration instruction Pᵢ*, resulting in an adapted or displaced vessel mask. The displaced vessel mask is then overlaid on the live image Lₖ to generate the corresponding overlay image.

This may be carried out analogously for all k=1, . . . , M.

In alternative embodiments, the live image is compared with the contrast-agent images in order to ascertain i*, as explained further below.

As already mentioned, the at least two X-ray images may be the at least two subtraction images. Again, in this case, the live images are not overlaid with the subtraction image that best fits the live image but instead with the shared mask that has been displaced as described. This has the advantage that hence, regardless of the current movement state, for instance in a respiratory cycle, the basis is always the same shared vessel mask, which in particular represents the contrast-agent filling state that has the largest total amount of contrast agent or represents the virtual contrast-agent filling state. Thus, an optimum overlay image may be generated for a plurality of consecutive live images even when movement occurs during the acquisition of the live images.

According to at least one embodiment, the registration instruction is determined for each of the provisional vessel masks depending on the shared vessel mask. For each of the at least two live images, one of the contrast-agent images is identified by comparing the particular live image with the at least two contrast-agent images. For each of the at least two live images, for the purpose of generating the overlay image, the shared vessel mask is registered with the particular live image by the registration instruction that corresponds to the identified contrast-agent image and is overlaid on the particular live image.

The above explanations for embodiments in which the live image is compared with the X-ray images may apply analogously.

In particular when there is provision for generating the subtraction images, the comparison of the live images with the contrast-agent images may be advantageous over a comparison of the live images with the subtraction images because the contrast-agent images, like the live images, represent not just the vascular structure but also the anatomical background.

According to at least one embodiment, the first MLM includes a CNN, for example a U-Net or a CNN designed on the basis of the U-Net, or a transformer network or a TransUNet or an ANN designed on the basis of the TransUNet or an MTM or an ANN designed on the basis of the MTM.

According to at least one embodiment, the at least two X-ray images are generated during administration of a contrast agent into the vascular structure.

The X-ray images may thereby be generated at different contrast-agent filling states.

According to at least one embodiment, the at least two contrast-agent images are generated during administration of the contrast agent into the vascular structure.

According to at least one embodiment, the at least two X-ray images represent the vascular structure during different movement states of the object.

For example, the different movement states may correspond to different movement states of a cyclical movement, for instance of a respiratory movement or of a cardiac movement. Other, in particular non-cyclical, movements of the object may also be involved, however.

According to at least one embodiment, at least one live image of the at least two live images represents an instrument for vascular intervention in the region to be depicted.

According to a further aspect, a data processing apparatus is specified. The data processing apparatus has at least one computing unit, which is configured to perform an X-ray imaging method.

In the present disclosure, the terms “data processing apparatus” and “at least one computing unit” may be used interchangeably. A computing unit may be understood to mean in particular a data processing unit which contains a processing circuit. In particular, the computing unit may thus process data for performing computing operations. These also include operations for performing indexed accesses to a data structure, for instance to a look-up table (LUT).

The computing unit may contain in particular one or more computers, one or more microcontrollers, and/or one or more integrated circuits, for example, one or more application-specific integrated circuits (ASIC), one or more field-programmable gate arrays (FPGA), and/or one or more systems on a chip (SoC). The computing unit may also contain one or more processors, for example, one or more microprocessors, one or more central processing units (CPU), one or more graphics processing units (GPU), and/or one or more signal processors, in particular one or more digital signal processors (DSP). The computing unit may also contain a physical or virtual interconnection of computers or other of the aforementioned units.

In various embodiments, the computing unit contains one or more hardware and/or software interfaces and/or one or more memory units.

A memory unit may be embodied as a volatile data storage medium, for example, as a dynamic random access memory (DRAM) or a static random access memory (SRAM), or as a non-volatile data storage medium, for example, as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM).

According to a further aspect, an X-ray imaging system is specified. The X-ray imaging system has a data processing apparatus as described herein. The X-ray imaging system has an X-ray source and an X-ray detector for generating the at least two live images and/or the at least two X-ray images and/or the at least one mask image and/or the at least two contrast-agent images.

According to at least one embodiment, the X-ray imaging system includes the display device, and the computing unit is configured to display the overlay images on the display device.

Further embodiments of the X-ray imaging system follow directly from the various embodiments of the X-ray imaging method, and vice versa. In particular, individual features and associated explanations and advantages relating to the various embodiments for the X-ray imaging method may be applied analogously to corresponding embodiments of the X-ray imaging system.

According to a further aspect, a computer program containing commands is specified. When the commands are executed by at least one computing unit, the commands cause the at least one computing unit to perform an X-ray imaging method.

For example, the commands may exist as program code. The program code may be provided, for example, as binary code or assembler and/or as source code of a programming language, for instance C, and/or as program script, for instance Python.

According to a further aspect, a computer-readable storage medium is defined, which stores a computer program.

The computer program and the computer-readable storage medium are each computer program products containing the commands.

Further features and combinations of features of the disclosure appear in the figures and the description of the figures and in the claims. In particular, further embodiments need not necessarily contain all the features of one of the claims. Further embodiments may have features and combinations of features that are not mentioned in the claims.

The disclosure is described in greater detail below with reference to specific embodiments and associated schematic drawings. In the figures, identical or functionally equivalent elements may be denoted by the same reference signs. The description of identical or functionally equivalent elements is not necessarily repeated when referring to different figures.

### BRIEF DESCRIPTION OF THE DRAWINGS

FIG. 1 depicts a schematic representation of an embodiment of an X-ray imaging system.

FIG. 2 depicts a flow diagram of an embodiment of an X-ray imaging method.

FIG. 3 depicts a flow diagram of a further embodiment of an X-ray imaging method.

FIG. 4 depicts a flow diagram of a further embodiment of an X-ray imaging method.

FIG. 5 depicts an embodiment of a convolutional neural network.

FIG. 6 depicts an exemplary embodiment of a convolutional neural network having a U-Net structure.

### DETAILED DESCRIPTION

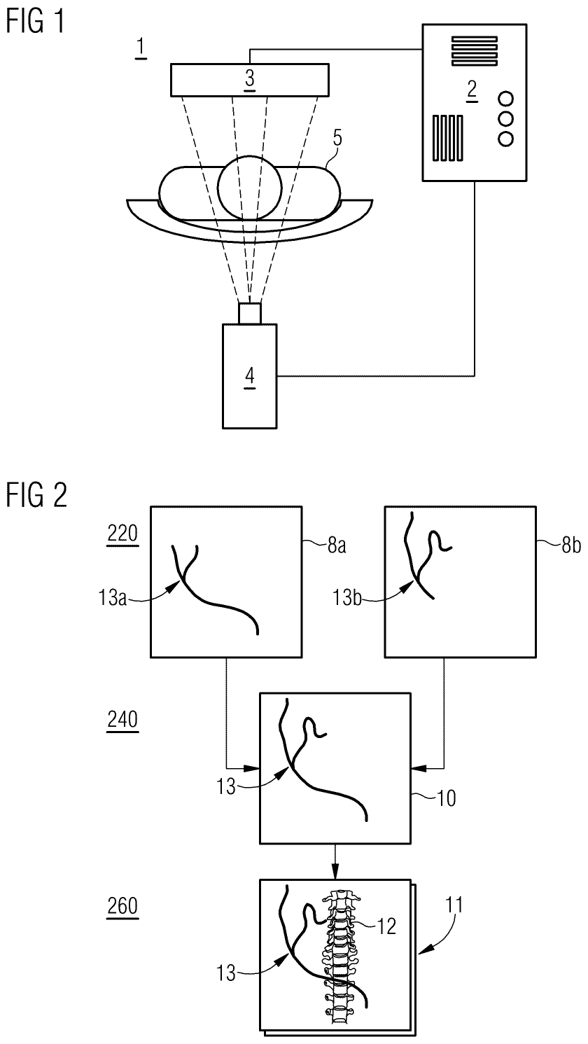

FIG. 1 shows schematically an embodiment of an X-ray imaging system 1. The X-ray imaging system 1 has an X-ray source 4 and an X-ray detector 3, and at least one computing unit 2, which is configured to perform an X-ray imaging method.

FIG. 2 shows a flow diagram of an embodiment of an X-ray imaging method.

In act 220, at least two X-ray images 8a, 8b of a region to be depicted in an object 5 are obtained. The at least two X-ray images 8a, 8b here correspond to different first acquisition timeframes and represent a vascular structure 13, 13a, 13b of the object 5 in different contrast-agent filling states. For example, the X-ray images 8a, 8b are generated by the X-ray imaging system 1 during administration of a contrast agent into the vascular structure 13, 13a, 13b, and correspond, for example, to different movement states of the object 5.

In act 240, a shared vessel mask 10, which represents the vascular structure 13, 13a, 13b, is generated on the basis of the at least two X-ray images 8a, 8b.

In act 260, at least two live images of the region to be depicted are obtained, which correspond to different second acquisition timeframes. In act 260, for each of the at least two live images, depending on the shared vessel mask 10, a corresponding overlay image 11 for displaying on a display device of the X-ray imaging system 1 is also generated, and displayed, for example, on the display device.

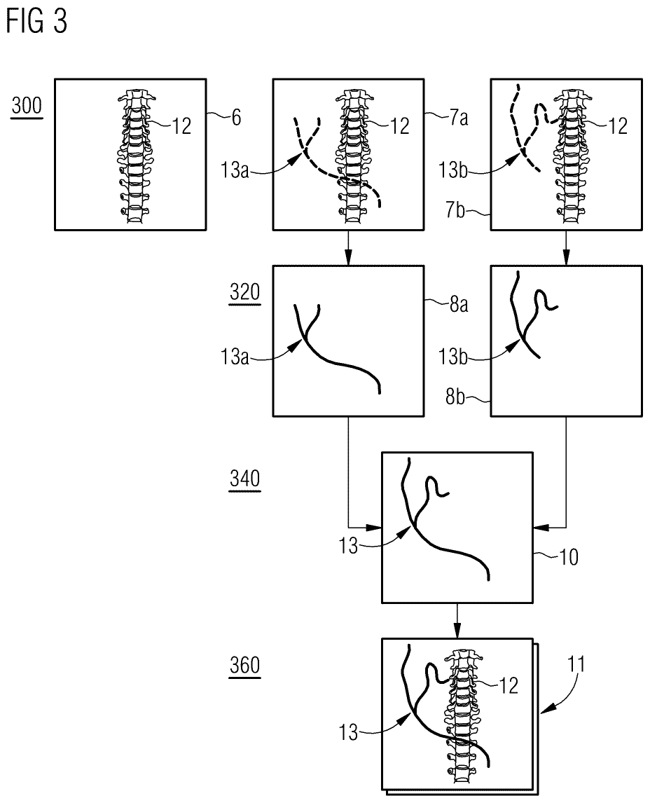

FIG. 3 shows a flow diagram of a further embodiment of an X-ray imaging method.

In this case, the at least two X-ray images 8a, 8b are generated as subtraction images 8a, 8b, in particular as part of DSA.

In act 300, at least one mask image 6 of the region to be depicted is obtained, and at least two contrast-agent images 7a, 7b of the region to be depicted are obtained, wherein the at least two contrast-agent images 7a, 7b represent the vascular structure 13, 13a, 13b in the different contrast-agent filling states.

In act 320, for each of the at least two contrast-agent images 7a, 7b, a subtraction image 8a, 8b is generated on the basis of the at least one mask image 6. For example, the mask image 6, which is generated without administration of contrast agent, is subtracted from each of the at least two contrast-agent images 7a, 7b in order to generate a corresponding subtraction image 8a, 8b. The mask image 6 shows, for example, an anatomical background 12 and the vascular structure 13, 13a, 13b, although the vascular structure 13, 13a, 13b is not represented in the mask image 6 in FIG. 3. For example, the anatomical background 12 is suppressed in the subtraction images 8a, 8b.

Alternatively, in act 300, at least two mask images 6 are obtained, wherein the at least two mask images 6 correspond to different further acquisition timeframes. The subtraction images 8a, 8b are generated on the basis of the at least two mask images 6 and the at least two contrast-agent images 7a, 7b.

For example, for each of the at least two contrast-agent images 7a, 7b a corresponding summation image (9a, 9b) is generated by weighted summation of the at least two mask images 6. For each of the at least two contrast-agent images 7a, 7b, the associated subtraction image 8a, 8b is generated by subtracting the associated summation image (9a, 9b) from the particular contrast-agent image 7a, 7b.

In act 340, a shared vessel mask 10, which represents the vascular structure 13, 13a, 13b, is generated on the basis of the at least two subtraction images 8a, 8b. This is done, for example, by applying a suitably trained MLM to input data that includes the subtraction images 8a, 8b.

In act 360, at least two live images of the region to be depicted are obtained, which correspond to different second acquisition timeframes. In act 360, for each of the at least two live images, depending on the shared vessel mask 10, a corresponding overlay image 11 for displaying on a display device of the X-ray imaging system 1 is also generated, and displayed, for example, on the display device.

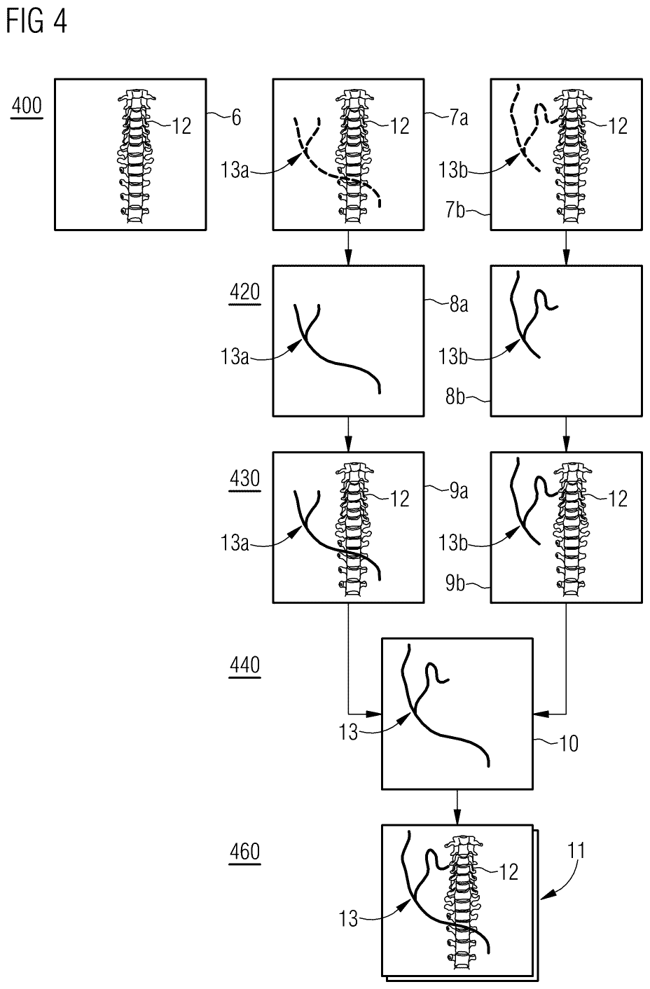

FIG. 4 shows a flow diagram of a further embodiment of an X-ray imaging method, which is based on the embodiment in FIG. 3, wherein at least two mask images 6 are obtained. The acts 400, 420, and 460 are equivalent to the act 300, 320, and 360, respectively. The generating of the shared vessel mask 10 differs in the method according to FIG. 3 from the method according to FIG. 4.

In the optional act 430, for each of the at least two subtraction images 8a, 8b, a corresponding summation image 9a, 9b is generated by weighted summation of the at least two mask images 6. The summation images 9a, 9b may be regarded here as modified contrast-agent images 7a, 7b, in each of which the vascular structure 13, 13a, 13b is enhanced. For each of the at least two contrast-agent images 7a, 7b, the associated subtraction image 8a, 8b is generated by subtracting the associated summation image 9a, 9b from the particular contrast-agent image 7a, 7b.

For each of the at least two subtraction images 8a, 8b, a provisional vessel mask is generated by applying a trained further MLM to further input data that contains the particular subtraction image 8a, 8b. Alternatively, for each of the at least two summation images 9a, 9b a provisional vessel mask is generated by applying the trained further MLM to further input data that contains the particular summation image 9a, 9b.

The further MLM is trained in particular for vessel extraction or vessel segmentation. Thus, the provisional vessel masks represent in particular the vascular structure 13, 13a, 13b extracted from the associated subtraction image 8a, 8b or summation image 9a, 9b. For example, the further MLM may be a CNN, in particular a U-Net, for vessel extraction.

The shared vessel mask 10 is selected in act 440 as one of the provisional vessel masks according to a defined rule. For example, this is done by selecting the provisional vessel mask that has the largest pixel L2 norm, i.e., the one that represents the contrast-agent filling state having the largest amount of contrast agent. Alternatively, other image metrics or methods based on machine learning may also be used for the selection, and/or provisional vessel masks may be combined in order to generate the shared vessel mask 10.

In act 460, at least two live images of the region to be depicted are obtained, which correspond to different second acquisition timeframes. In act 460, for each of the at least two live images, depending on the shared vessel mask 10, a corresponding overlay image 11 for displaying on a display device of the X-ray imaging system 1 is also generated, and displayed, for example, on the display device.

In certain embodiments, a registration instruction is determined for each of the provisional vessel masks depending on the shared vessel mask 10, for instance by determining a displacement vector between the shared vessel mask 10 and the particular provisional vessel mask that maximizes the NCC between the shared vessel mask 10 and the particular provisional vessel mask.

For example, this is done by performing an exhaustive search on an image pyramid of a plurality of resolutions inside a region of the movement of the object 5. Alternatively, registration techniques based on gradient optimization or based on machine learning may be used. The shared vessel mask 10 is accordingly calculated by pixel-by-pixel displacement using the optimum displacement vector that was found.

For example, for each of the at least two live images, a comparison of the particular live image with the at least two contrast-agent images 7a, 7b is performed in order to identify one of the contrast-agent images 7a, 7b. This is performed, for example, by calculating the NCC between the particular live image and the particular contrast-agent image 7a, 7b, and identifying the contrast-agent image 7a, 7b that has the greatest NCC with the particular live image.

For example, for the purpose of generating the associated overlay image 11, the shared vessel mask 10 is registered with the particular live image by the registration instruction that corresponds to the identified contrast-agent image 7a, 7b, and overlaid on the particular live image.

In various embodiments of the X-ray imaging method, a vessel mask sufficiently filled with contrast agent is provided for each live image, in particular for each movement state, for instance each respiratory phase. In addition, an adapted acquisition with extended administration of contrast agent may be performed if applicable.

Various embodiments have the advantage that the vessel masks obtained by an MLM do not exhibit noise and have fewer anatomical artifacts than the subtraction images, which results in improved image quality in the overlay images.

In various embodiments, the registration is carried out only in relation to the provisional vessel masks that do not contain any bone structures and are independent of the movement. This may improve the accuracy of known motion compensation functions.

In various embodiments, the displaced vessel masks may be generated within a few seconds, and the overlay images calculated in real time. The most time-consuming computation act may be the registration, although this may easily have a parallel implementation using GPUs.

An MLM as used in some embodiments may be an ANN, for example, a CNN.

A CNN is an ANN that uses in at least one of its layers a convolution operation instead of a general matrix multiplication. These layers are also referred to as convolutional layers. In particular, a convolutional layer performs a dot product of one or more convolution kernels with the input data of the convolutional layer, where the entries in the one or more convolution kernels are parameters or weights that may be adapted by training. In particular, the Frobenius inner product and the ReLU activation function may be used. A convolutional neural network may include additional layers, for instance pooling layers, fully connected layers and/or normalization layers.

The use of convolutional neural networks allows very efficient processing of the input because a convolution operation based on different kernels may extract different image features, and therefore the relevant image features may be determined during the training by adapting the weights of the convolution kernel. Moreover, as a result of the shared use of the weights in the convolution kernels, fewer parameters have to be trained, which prevents overfitting in the training phase and allows faster training or more layers in the network, thereby improving the performance of the network.

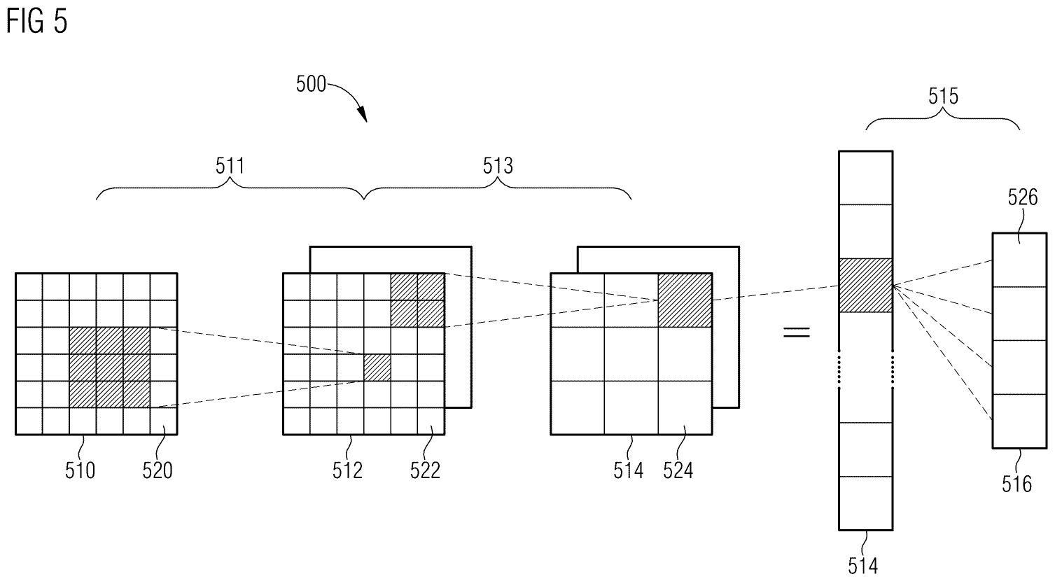

FIG. 5 shows an embodiment of a convolutional neural network 500. In the embodiment shown, the convolutional neural network 500 includes an input node layer 510, a convolutional layer 511, a pooling layer 513, a fully connected layer 514, and an output node layer 516, and also hidden node layers 512, 514. Alternatively, the convolutional neural network 500 may also include a plurality of convolutional layers 511, a plurality of pooling layers 513, and/or a plurality of fully connected layers 515, and also other types of layers. The layers may be chosen to be in any order, although normally fully connected layers 515 are used as the last layers before the output layer 516.

In a convolutional neural network 500, the nodes 520, 522, 524 in a node layer 510, 512, 514 may be viewed in particular as a d-dimensional matrix or as a d-dimensional image. In particular, in the two-dimensional case, the value of the node 520, 522, 524 with indices i and j in the n-th node layer 510, 512, 514 may be denoted by x(n)[i, j]. The arrangement of the nodes 520, 522, 524 in a node layer 510, 512, 514 as such has no effect, however, on the calculations performed inside the convolutional neural network 500, because these are given solely by the structure and the weights of the edges.

A convolutional layer 511 is a connecting layer between a preceding node layer 510 containing node values x(n−1) and a following node layer 512 containing node values x(n). A convolutional layer 511 is characterized in particular by the structure and the weights of the ingoing edges, which form a convolution operation based on a certain number of kernels. In particular, the structure and the weights of the edges of the convolutional layer 511 may be selected such that the values x(n) of the nodes 522 in the following convolutional layer 512 are calculated as a convolution x(n)=K*x(n−1) based on the values x(n−1) of the nodes 520 in the preceding layer 510, where the convolution * in the two-dimensional case is defined as:

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here, the kernel K is a d-dimensional matrix, in the present example a two-dimensional matrix, which may be small in comparison with the number of nodes 520, 522, for instance a 3×3 matrix or a 5×5 matrix. This means in particular that the weights of the edges in the convolutional layer 511 are not independent but are selected such that they create the stated convolution equation. In particular, there are only 9 independent weights for a kernel that is a 3×3 matrix, where each entry in the kernel matrix corresponds to an independent weight, regardless of the number of nodes 520, 522 in the preceding node layer 510 and the following node layer 512.

In certain examples, convolutional neural networks 500 use node layers 510, 512, 514 having a multiplicity of channels, in particular as a result of using a multiplicity of kernels in the convolutional layers 511. In these cases, the node layers may be viewed as (d+1)-dimensional matrices, where the first dimension is the index of the channels. The effect of a convolutional layer 511 is then defined in a two-dimensional example as:

$${x}_{b}^{( n )} [ i , j ] = {\sum }_{a} ( {K}_{a , b} * {x}_{a}^{( n - 1 )} [ i , j ] = {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ,$$

where xₐ⁽ⁿ⁾ corresponds to the a-th channel of the preceding node layer 510, x_{b}⁽ⁿ⁾, corresponds to the b-th channel of the subsequent node layer 512, and K_{a,b} corresponds to one of the kernels.

If a convolutional layer 511 acts on a preceding node layer 510 having A-channels and outputs a subsequent node layer 512 having B-channels, there are A·B independent d-dimensional kernels K_{a,b}.

In certain examples, activation functions may be used in convolutional neural networks 500. In this embodiment, ReLU (rectified linear unit) is used, with R(z)=max(0, z), so that the effect of the convolutional layer 511 in the two-dimensional example is:

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] )$$

It is also possible to use other activation functions, for instance ELU (exponential linear unit), leaky ReLU, sigmoid, tanh, or softmax.

In the embodiment shown, the input layer 510 includes 36 nodes 520, which are arranged in a two-dimensional 6×6 matrix. The first hidden node layer 512 contains 72 nodes 522, which are arranged as two-dimensional 6×6 matrices, with each of the two matrices being the result of convolving the values in the input layer with a 3×3 kernel in the convolutional layer 511. As an equivalent, the nodes 522 in the first hidden node layer 512 may be interpreted as a three-dimensional 2×6×6 matrix, where the first dimension corresponds to the channel dimension.

An advantage of using convolutional layers 511 is that a spatially local correlation in the input data may be exploited by imposing a local connectivity pattern between the nodes in neighboring layers, in particular by each node being connected just to a small region of the nodes in the previous layer.

A pooling layer 513 is a connecting layer between a previous node layer 512 containing node values x(n−1) and a subsequent node layer 514 containing node values x(n). A pooling layer 513 may be characterized in particular by the structure and the weights of the edges and by the activation function, which form a pooling operation based on a non-linear pooling function f. For example, in the two-dimensional case, the values x(n) of the nodes 524 in the subsequent node layer 514 are calculated on the basis of the values x(n−1) of the nodes 522 in the anterior node layer 512 as follows:

$${x}_{b}^{( n )} [ i , j ] = f ( {x}_{b}^{( n - 1 )} [ i {d}_{1} , {jd}_{2} ] , … , {x}_{b}^{( n - 1 )} [ ( i + 1 ) {d}_{1} - 1 , ( j + 1 ) {d}_{2} - 1 ] ) .$$

In other words, using a pooling layer 513 may reduce the number of nodes 522, 524 by replacing a number of d1-d2 adjacent nodes 522 in the previous node layer 512 with a single node 522 in the subsequent node layer 514, which is calculated as a function of the values of the number of adjacent nodes. In particular, the pooling function f may be the max function, the mean value, or the L2 norm. In particular, the weights of the ingoing edges are fixed for a pooling layer 513 and not modified by the training.

The advantage of using a pooling layer 513 is that it reduces the number of nodes 522, 524 and the number of parameters. This results in a reduction in the calculation effort in the network and/or acts as a check on overfitting.

In the embodiment shown, the pooling layer 513 is a max pooling layer, in which four adjacent nodes are replaced by just one node, where the value is the maximum of the values of the four adjacent nodes. The max pooling is applied to each d-dimensional matrix of the previous layer. In this embodiment, the max pooling is applied to each of the two-dimensional matrices, thereby reducing the number of nodes from 72 to 18.

In certain examples, the last layers of a convolutional neural network 500 may be fully connected layers 515. A fully connected layer 515 is a connecting layer between a previous node layer 514 and a subsequent node layer 516. A fully connected layer 513 may be characterized in that a plurality of, in particular all, edges between the nodes 514 in the previous node layer 514 and the nodes 516 in the subsequent node layer are present, and the weight of each of these edges may be adjusted individually.

In this embodiment, the nodes 524 in the preceding node layer 514 of the fully connected layer 515 are represented both as two-dimensional matrices and also additionally as non-related nodes, which are shown as a line of nodes, where the number of nodes has been reduced for better visualization. This process is also referred to as flattening. In this embodiment, the number of nodes 526 in the subsequent node layer 516 of the fully connected layer 515 is less than the number of nodes 524 in the previous node layer 514. Alternatively, the number of nodes 526 may also be the same or larger.

In addition, in this embodiment, the softmax activation function is used in the fully connected layer 515. By applying the softmax function, the sum of the values of all the nodes 526 in the output layer 516 equals 1, and all the values of all the nodes 526 in the output layer 516 are real numbers between 0 and 1. In particular, when using the convolutional neural network 500 for categorizing input data, the values of the output layer 516 may be interpreted as the probability of the input data falling into one of the different categories.

In particular, convolutional neural networks 500 may be trained on the basis of the backpropagation algorithm. Regularization methods may be employed in order to avoid overfitting, for example dropout of nodes 520, . . . , 524, stochastic pooling, the use of artificial data, weight decay based on the L1 or L2 norm, or max norm constraints.

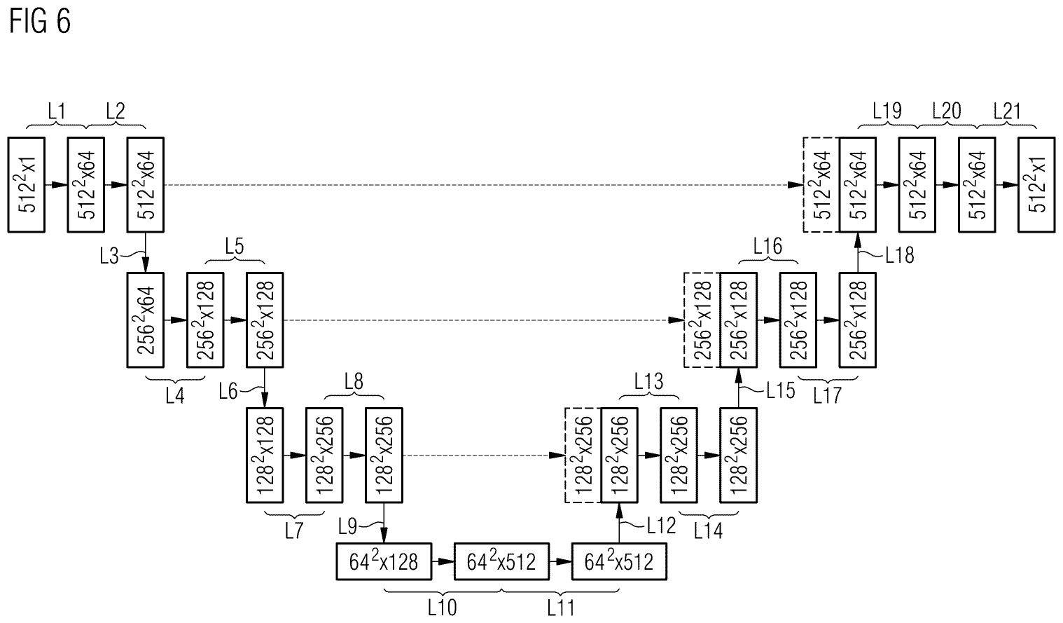

In the example of FIG. 6, the MLM is a CNN with a U-Net structure. In the example shown, the input data for the CNN is a two-dimensional medical image containing 512×512 pixels, where each pixel contains an intensity value. The CNN includes convolutional layers, which are represented by continuous horizontal arrows, pooling layers, which are represented by continuous downward-pointing arrows, and upsampling layers, which are represented by continuous upward-pointing arrows. The number of nodes in each case is shown in the boxes. Within the U-Net structure, the input images are first downsampled, in particular by reducing the size of the images and increasing the number of channels. Then they are upsampled, in particular by increasing the size of the images and reducing the number of channels in order to generate a transformed image.

All except the last convolutional layers L1, L2, L4, L5, L.7, L8, L10, L11, L13, L14, L16, L17, L19, L20 use 3×3 kernels with a padding of 1, the ReLU activation function and a number of filters or convolution kernels equal to the number of channels of the respective node layers, as shown in FIG. 6. The last convolutional layer uses a 1×1 kernel without padding and the ReLu activation function.

The pooling layers L3, L6, L9 are max pooling layers, which replace four adjacent nodes with just one node, where the value is the maximum of the values of the four adjacent nodes. The upsampling layers L12, L15, L18 are transposed convolutional layers using 3×3 kernels and stride 2, thereby effectively quadrupling the number of nodes. The dashed horizontal arrows correspond to concatenation operations in which the output of a convolutional layer L2, L5, L8 in the downsampling branch of the U-Net structure is used as additional inputs for a convolutional layer L13, L16, L19 in the upsampling branch of the U-Net structure. This additional input data is treated as additional channels in the input node layer for the convolutional layer L13, L16, L19 in the upsampling branch.

A database containing 500 first medical images was used to train the CNN, where the respective segmentation masks were created on the basis of annotations by expert radiologists. In particular, the experts ascertained for each of the 500 first medical images a segmentation mask for a structure of interest, where a value of 1 was assigned to the pixels corresponding to the structure of interest, and a value of 0 to the pixels not corresponding to the structure of interest. The database was split into training data (320 datasets), validation data (80 datasets) and test data (100 datasets). The backpropagation algorithm, based on a binary cross-entropy cost function, was used to train the CNN:

$$L ( x , y ) = {\sum }_{i} {\sum }_{j} B C E ( y [ i , j ] , M ( x ) [ i , j ] )$$

$$with BCE ( a , b ) := - a log ( b ) ( b ) - ( 1 - a ) log ( 1 - b ) ,$$

where x denotes a first medical image, y defines the corresponding segmentation mask that was created by the radiology expert, and M(x) denotes the result of applying the CNN to the first medical input image x. Alternatively, other cost functions such as weighted binary cross-entropy, focal loss, or dice loss may also be used.

On the basis of the validation set of 80 datasets and the corresponding annotations, the model with the best performance was selected from a plurality of machine-learning models (having different hyperparameters, for instance number of layers, size and number of kernels, padding, et cetera). The specificity and sensitivity are ascertained on the basis of the test set, which included 100 datasets and the corresponding annotations.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

Independent of the grammatical term usage, individuals with male, female or other gender identities are included within the term.

1. An X-ray imaging method comprising:
obtaining at least two X-ray images of a region to be depicted in an object, wherein the at least two X-ray images correspond to different first acquisition timeframes and represent a vascular structure of the object in different contrast-agent filling states;
generating a shared vessel mask, which represents the vascular structure, based on the at least two X-ray images;
obtaining at least two live images of the region to be depicted, wherein the at least two live images correspond to different second acquisition timeframes; and
generating, for each live image of the at least two live images, depending on the shared vessel mask, a corresponding overlay image for displaying on a display device.

2. The X-ray imaging method of claim 1, further comprising:
obtaining at least one mask image of the region to be depicted;
obtaining at least two contrast-agent images of the region to be depicted are obtained, wherein the at least two contrast-agent images represent the vascular structure in the different contrast-agent filling states; and
generating, for each contrast-agent image of the at least two contrast-agent images, a subtraction image based on the at least one mask image,
wherein the at least two X-ray images are given by the subtraction images.

3. The X-ray imaging method of claim 2, wherein the at least one mask image comprises at least two mask images,
wherein the at least two mask images correspond to different further acquisition timeframes,
wherein, for each contrast-agent image of the at least two contrast-agent images, a corresponding summation image is generated by weighted summation of the at least two mask images, and
wherein, for each contrast-agent image of the at least two contrast-agent images, the associated subtraction image is generated by subtracting the associated summation image from the particular contrast-agent image.

4. The X-ray imaging method of claim 1, wherein the generating of the shared vessel mask comprises applying a trained first machine-learning model to first input data, which depends on the at least two X-ray images.

5. The X-ray imaging method of claim 4, wherein the trained first machine-learning model comprises a convolutional neural network or a transformer network.

6. The X-ray imaging method of claim 2, further comprising:
generating, for each X-ray image of the at least two X-ray images, a provisional vessel mask by applying a trained first machine-learning model to first input data, which depends on the particular X-ray image,
wherein the shared vessel mask is generated depending on the provisional vessel masks.

7. The X-ray imaging method of claim 6, wherein the shared vessel mask is selected as one of the provisional vessel masks according to a defined rule, or
wherein the shared vessel mask is generated by applying a trained second machine-learning model to second input data, which depends on the provisional vessel masks.

8. The X-ray imaging method of claim 6, further comprising:
determining a registration instruction for each provisional vessel mask of the provisional vessel masks depending on the shared vessel mask;
determining a registration instruction for each live image of the at least two live images;
identifying a contrast-agent image of the contrast-agent images by comparing the particular live image with the at least two contrast-agent images;
registering, for the generating of the overlay image, the shared vessel mask with the particular live image by the registration instruction that corresponds to the identified contrast-agent image; and
overlaying the registered shared vessel mask on the particular live image.

9. The X-ray imaging method of claim 1, further comprising:
generating, for each X-ray image of the at least two X-ray images, a provisional vessel mask by applying a trained first machine-learning model to first input data, which depends on the particular X-ray image,
wherein the shared vessel mask is generated depending on the provisional vessel masks.

10. The X-ray imaging method of claim 9, wherein the shared vessel mask is selected as one of the provisional vessel masks according to a defined rule, or
wherein the shared vessel mask is generated by applying a trained second machine-learning model to second input data, which depends on the provisional vessel masks.

11. The X-ray imaging method of claim 9, further comprising:
determining a registration instruction for each provisional vessel mask of the provisional vessel masks depending on the shared vessel mask;
determining a registration instruction for each live image of the at least two live images;
identifying an X-ray image of the at least two X-ray images by comparing the particular live image with the at least two X-ray images;
registering, for the generating of the overlay image, the shared vessel mask with the particular live image by the registration instruction that corresponds to the identified X-ray image; and
overlaying the registered shared vessel mask on the particular live image.

12. The X-ray imaging method of claim 9, wherein the trained first machine-learning model comprises a convolutional neural network or a transformer network.

13. The X-ray imaging method of claim 1, wherein the at least two X-ray images are generated during administration of a contrast agent into the vascular structure.

14. The X-ray imaging method of claim 1, wherein the at least two X-ray images represent the vascular structure during different movement states of the object.

15. The X-ray imaging method of claim 1, wherein at least one live image of the at least two live images represents an instrument for vascular intervention in the region to be depicted.

16. A data processing apparatus comprising:
at least one computing unit configured to:
obtain at least two X-ray images of a region to be depicted in an object, wherein the at least two X-ray images correspond to different first acquisition timeframes and represent a vascular structure of the object in different contrast-agent filling states;
generate a shared vessel mask, which represents the vascular structure, based on the at least two X-ray images;
obtain at least two live images of the region to be depicted, wherein the at least two live images correspond to different second acquisition timeframes; and
generate, for each live image of the at least two live images, depending on the shared vessel mask, a corresponding overlay image for displaying on a display device.

17. An X-ray imaging system comprising:
an X-ray source and an X-ray detector for generating at least two live images; and
a data processing apparatus having at least one computing unit configured to:
obtain at least two X-ray images of a region to be depicted in an object, wherein the at least two X-ray images correspond to different first acquisition timeframes and represent a vascular structure of the object in different contrast-agent filling states;
generate a shared vessel mask, which represents the vascular structure, based on the at least two X-ray images;
obtain the at least two live images of the region to be depicted, wherein the at least two live images correspond to different second acquisition timeframes; and
generate, for each live image of the at least two live images, depending on the shared vessel mask, a corresponding overlay image for displaying on a display device.

18. The X-ray imaging system of claim 17, further comprising:
the display device configured to display the corresponding overlay image.
